(19) United States
(12) Patent Application Publication
(10) Pub. No.: US 2026/0364133 A1
(43) Pub. Date: Nov. 19, 2026

(54) BELLFLOWER-ROOT POWDER WITH ENHANCED SAPONIN AND FLAVONOID CONTENTS AND IMPROVED STORAGE STABILITY, AND METHOD FOR MANUFACTURING THE SAME

(71) Applicant: SU.KOREA Co., Ltd., Gwangmyeong-si (KR)

(72) Inventor: Jeong Seon SHIN, Gwangmyeong-si (KR)

(21) Appl. No.: 19/403,077

(22) Filed: Nov. 27, 2025

(30) Foreign Application Priority Data
May 19, 2025 (KR) 10-2025-0064412

(51) Int. Cl. A61K 36/346; A61K 36/752; A61K 36/899; A61K 47/36; C12N 9/42

(52) U.S. Cl. CPC A61K 36/346; A61K 36/752; A61K 36/899; A61K 47/36; C12N 9/2437; A61K 2236/13; A61K 2236/15; A61K 2236/19; A61K 2236/333; A61K 2236/51; C12Y 302/01004; C12Y 302/01015

(57) ABSTRACT

The present invention, in preparing a medicinal Platycodon root powder, (i) decomposes the cell wall by enzyme treatment, (ii) forms an antimicrobial and moisture-protective layer through coating with grapefruit seed extract and fructooligosaccharide, and (iii) recovers and concentrates residual saponins through low-temperature extraction and re-mixing, thereby providing a powder having high saponin and flavonoid contents and excellent storage stability without high-heat processing. This powder has high utility as a material for medicinal Platycodon root syrup, powdered beverages, and health functional food ingredients.

### CROSS-REFERENCE TO RELATED APPLICATIONS

This application claims the priority of the Korean Patent Applications No. 10-2025-0064412, filed on May 19, 2025, in the Korean Intellectual Property Office. The entire disclosures of all these applications are hereby incorporated by reference.

### TECHNICAL FIELD

It relates to a medicinal Platycodon root powder having enhanced saponin and flavonoid contents and excellent storage stability, and a method for preparing the same, and is for preparing a medicinal Platycodon root powder for producing medicinal Platycodon root syrup.

### BACKGROUND ART

Medicinal Platycodon root has been widely used since ancient times for its antitussive, expectorant, and anti-inflammatory effects, and its major pharmacological components are known to be triterpenoid saponins (such as platycodin D) and various flavonoids. However, the raw Platycodon root possesses a distinct bitter taste and a strong root odor, resulting in low palatability, and it is vulnerable to heat and oxygen, such that saponins are easily decomposed and browning, caking, and microbial proliferation occur frequently when only general hot-air drying or high-temperature concentration processes are employed. In order to solve these problems, individual techniques such as enzymatic pretreatment, low-temperature extraction, and preservative addition have been proposed, but an integrated process that simultaneously satisfies enhancement of active components and storage stability has not been presented.

### PRIOR ART DOCUMENTS

### Patent Documents

(Patent Document 1) Korean Registered Patent No. 10-2611611 (registered on Dec. 5, 2023)

### DISCLOSURE OF THE INVENTION

### Technical Problem

Various embodiments of the present disclosure are intended to solve the above problems, and to provide a medicinal Platycodon root powder that maximizes the content of major active components such as Platycodon root saponins, flavonoids, and inulin while minimizing their loss, maintains quality for a long period by suppressing caking, discoloration, and microbial growth even under low-sugar and low-moisture conditions, and exhibits excellent palatability by alleviating bitterness and root odor.

The problems to be solved by the present invention are not limited to those mentioned above, and other problems not mentioned will be clearly understood by a person of ordinary skill in the art from the description below.

### Technical Solution

An embodiment of the present invention for solving the above-described problems provides a method for preparing a medicinal Platycodon root powder comprising the following steps.

First, the raw medicinal Platycodon root is washed and dehydrated, and then dried with hot air at 45 °C or lower to adjust the moisture content to 10 wt % or less. Next, the dried material is subjected to a first pulverization to 20 mesh or less. The pulverized powder is mixed with distilled water at a ratio of 1:3 (w/v) to form a slurry, and 0.5 wt % cellulase and 0.3 wt % pectinase are added and reacted at 50 °C and pH 5.0 for 90 minutes, followed by freeze-drying at −40 °C and 10 Pa for 48 hours to obtain an enzyme-treated powder. In the next step, grapefruit seed extract and fructooligosaccharide are mixed at a solids ratio of 1:1 (w/w) to prepare a homogeneous solution at 45 °C, which is then spray-coated onto a drum rotating at 15 rpm so that 30 wt % is attached relative to the total powder, and then dried at 45 °C and −0.08 MPa for 1.5 hours to obtain a premix-coated powder. As necessary, the coated powder may be subjected to low-temperature extraction with a mixed solvent of 50% ethanol and water at 40 °C for 60 minutes, concentrated to 25 Brix at 45 °C and −0.08 MPa, and dried by a spray-drying method, and the resulting concentrated powder is re-mixed with the primary powder at a ratio of 1:9 to prepare a low-temperature-extraction-remixed powder. Finally, the remixed powder is subjected to a second fine pulverization to 100 mesh or less, and is formulated with 92 wt % medicinal Platycodon root powder, 5 wt % glutinous rice powder (domestically produced), 1 wt % malt (domestically produced), 1 wt % grapefruit seed extract, and 1 wt % fructooligosaccharide, and then filled into brown glass bottles in units of 120 g and packaged.

Other specific details of the present invention are included in the detailed description and drawings.

### Advantageous Effects

According to the present invention, by applying a continuous process of enzyme treatment, premix coating, and low-temperature extraction and re-mixing, the contents of saponins, flavonoids, and inulin, which are major active components in the medicinal Platycodon root powder, can be improved by about 1.8 times, 2.8 times, and 2.3 times, respectively, compared to conventional processes. Accordingly, the functionality of the medicinal Platycodon root powder is greatly enhanced.

In addition, caking and discoloration can be effectively suppressed even under low-sugar and low-moisture conditions, and appearance quality and microbial safety are excellently maintained during long-term storage for more than 6 months even in an environment of 40 °C and 75% RH. Accordingly, the commercial value of the product is improved, and consumer palatability is also enhanced.

In addition, bitterness and root odor are alleviated, reducing aversion during intake, and antimicrobial properties are imparted through grapefruit seed extract-based coating, thereby enhancing the effect of suppressing microbial proliferation. Thus, a hygienic and stable medicinal Platycodon root powder product can be provided.

The effects of the present invention are not limited to the effects mentioned above, and other effects not mentioned will be clearly understood by a person of ordinary skill in the art from the description below.

### BRIEF DESCRIPTION OF THE DRAWINGS

Various aspects are described with reference to the drawings, where similar reference numerals are used collectively to refer to similar components. In the following embodiments, for purposes of illustration, numerous specific details are presented to provide a thorough understanding of one or more aspects. However, it will be apparent that such aspect(s) may be implemented without these specific details.

Referring to FIG. 1, a flowchart of the method for preparing a Platycodon root active-component-enhanced powder for preparing medicinal Platycodon root syrup according to the present invention is illustrated.

FIG. 2 illustrates the specific steps of the enzyme treatment step, the premix coating step, and the low-temperature extraction and re-mixing step described in FIG. 1.

### MODE FOR CARRYING OUT THE INVENTION

Various embodiments and/or aspects are now disclosed with reference to the drawings. In the following description, for purposes of explanation, numerous specific details are disclosed to assist in providing an overall understanding of one or more aspects. However, it will also be appreciated by those of ordinary skill in the art that such aspect(s) may be practiced without these specific details. The following description and the accompanying drawings describe in detail certain exemplary aspects of one or more aspects. However, these aspects are illustrative, and various ones among the principles of the various aspects may be utilized, and the described explanations are intended to include all such aspects and their equivalents. Specifically, as used in the present specification, terms such as “embodiment,” “example,” “aspect,” and “illustrative” do not necessarily indicate that any described aspect or design is preferred or advantageous over other aspects or designs.

Hereinafter, the same reference numerals are assigned to the same or similar components regardless of the drawing symbols, and redundant descriptions thereof are omitted. In addition, in describing the embodiments disclosed in the present specification, detailed descriptions of related known technologies are omitted when it is determined that such detailed descriptions may obscure the gist of the embodiments disclosed in the present specification. Further, the accompanying drawings are merely for facilitating understanding of the embodiments disclosed in the present specification, and the technical spirit disclosed in the present specification is not limited by the accompanying drawings.

Although terms such as first, second, and the like are used to describe various elements or components, it is of course that these elements or components are not limited by these terms. These terms are merely used to distinguish one element or component from another element or component. Therefore, it is also understood that the first element or component mentioned below may be the second element or component within the technical spirit of the present invention.

Unless otherwise defined, all terms (including technical and scientific terms) used in the present specification may be used in meanings commonly understood by those of ordinary skill in the technical field to which the present invention pertains. In addition, terms defined in generally used dictionaries are not interpreted ideally or excessively unless clearly and specifically defined otherwise.

Furthermore, the term “or” is intended to mean an inclusive “or,” rather than an exclusive “or.” That is, unless otherwise specified or clear from the context, “X uses A or B” is intended to mean any of the naturally inclusive substitutions. That is, “X uses A or B” applies to any of the cases where X uses A; X uses B; or X uses both A and B. In addition, the term “and/or” as used in the present specification is to be understood as referring to and including all possible combinations of one or more of the listed associated items.

In addition, the terms “comprises” and/or “comprising” are to be understood as indicating the presence of the corresponding feature and/or component, but not excluding the presence or addition of one or more other features, components, and/or groups thereof. Further, unless otherwise specified or unless the context clearly indicates that a singular form is intended, the singular in the present specification and the claims is to be interpreted as generally meaning “one or more.”

The objects and effects of the present invention, and the technical configurations for achieving them, will become clear with reference to the embodiments described in detail below together with the accompanying drawings. In describing the present invention, detailed descriptions of known functions or configurations will be omitted when it is determined that such detailed descriptions may unnecessarily obscure the gist of the present invention. In addition, the terms described below are terms defined in consideration of the functions in the present invention, and these may vary depending on the intention or customary practice of the user or operator.

However, the present invention is not limited to the embodiments disclosed below, and may be implemented in various different forms. These embodiments are merely provided to make the present invention complete and to fully inform those of ordinary skill in the art of the scope of the disclosure, and the present invention is defined only by the scope of the claims. Therefore, the definition should be made based on the contents throughout the entire specification.

Referring to FIG. 1, the method for preparing a Platycodon root active-component-enhanced powder for preparing medicinal Platycodon root syrup according to the present invention may include a washing and dehydrating step (S110), a low-temperature drying step (S120), a first pulverizing step (S130), an enzyme treatment step (S140), a premix coating step (S150), a low-temperature extraction and re-mixing step (S160), a second fine-pulverizing step (S170), and a packaging step (S180).

First, in the washing and dehydrating step (S110), the medicinal Platycodon root (or medicinal Platycodon root root) as a raw material may be washed with running water, and then dehydrated for 3 minutes using a spin dryer to remove surface moisture. Here, the spin dryer is a device that removes moisture by generating centrifugal force through high-speed rotation, and can efficiently remove surface moisture after washing. In the present invention, by dehydrating for 3 minutes at a rotation speed of, for example, about 2,000 rpm, excessive moisture can be rapidly removed while minimizing tissue damage of the medicinal Platycodon root.

In the low-temperature drying step (S120), the medicinal Platycodon root washed and dehydrated in step S110 is dried with hot air at 45 °C or lower for 14 hours or more so that the final moisture content becomes 10 wt % or less. Preferably, by drying in an environment with a relative humidity of 40% or less, thermal denaturation of flavonoids can be minimized.

In the first pulverizing step (S130), the medicinal Platycodon root dried in step S120 may be pulverized to 20 mesh or less. For example, the dried medicinal Platycodon root may be pulverized using a pin mill. The pulverized powder may undergo a sieving process in order to improve enzyme reaction and extraction efficiency. Here, the pin mill is a device that finely pulverizes raw material by utilizing impact force and shear force generated between pins attached to a rotor (or disc) rotating at high speed and pins fixed inside the housing. This process has high pulverizing efficiency and relatively low thermal load due to the short residence time, and is therefore suitable for pulverizing medicinal plant materials such as medicinal Platycodon root, which have high temperature sensitivity.

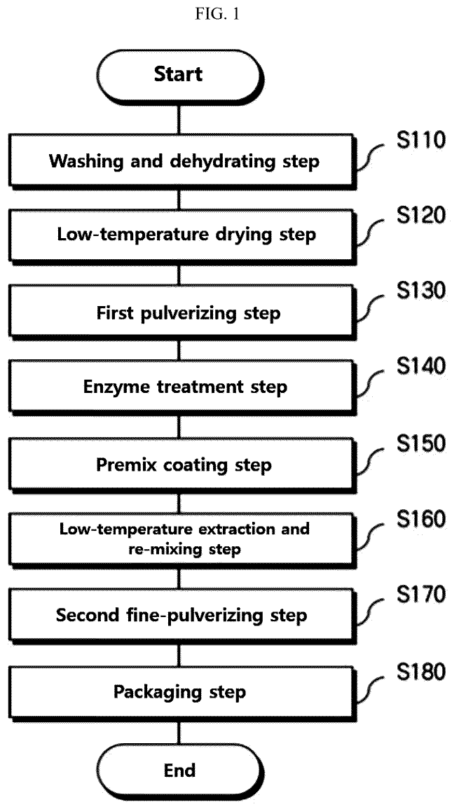

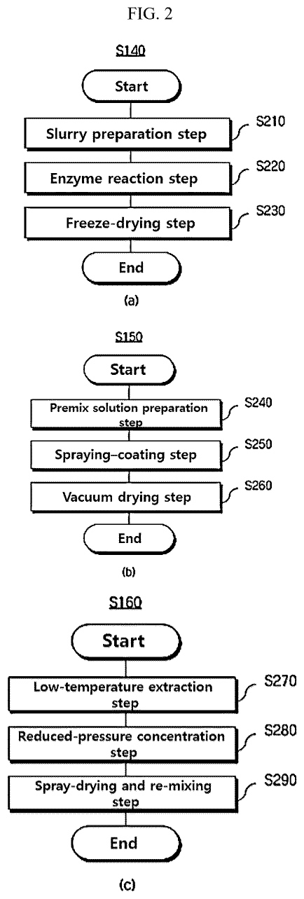

Next, the enzyme treatment step (S140), referring to FIG. 2 (a), may include a slurry preparation step (S210), an enzyme reaction step (S220), and a freeze-drying step (S230).

In the slurry preparation step (S210), the powder and distilled water may be mixed at 1:3 (w/v) to prepare a slurry. The enzyme reaction step (S220) may decompose the cell wall of the medicinal Platycodon root by adding 0.5 wt % cellulase and 0.3 wt % pectinase, and reacting at 50 °C and pH 5.0 for 90 minutes. Thereafter, in the freeze-drying step (S230), the slurry reacted in the enzyme reaction step (S220) may be freeze-dried at −40 °C and 10 Pa for 48 hours to prepare an enzyme-treated powder having a moisture content of 5 wt % or less. Here, freeze-drying is a process that maintains low-temperature and low-pressure conditions so that water sublimates directly from a solid state to a gaseous state. By applying −40 °C and 10 Pa in the present invention, denaturation of the active components in the reacted medicinal Platycodon root slurry is minimized, and the moisture content can be reduced to 5 wt % or less after 48 hours of drying.

Next, the premix coating step (S150), referring to FIG. 2 (b), may include a premix solution preparation step (S240), a spraying-coating step (S250), and a vacuum drying step (S260).

In the premix solution preparation step (S240), grapefruit seed extract and fructooligosaccharide may be mixed at a solids ratio of 1:1 (w/w) to prepare a homogeneous solution. For example, to prepare a premix coating solution, 150 g of grapefruit seed extract (GSE) solids and 150 g of fructooligosaccharide (FOS) solids may be targeted. To this end, 600 g of GSE liquid (25% solids, containing 150 g of GSE solids) and about 154.6 g of FOS P (97% solids, containing about 150 g of FOS solids) are used, and about 245.4 g of purified water is added thereto, followed by high-speed homogenization at 45 °C (3,000 rpm, 3 min) to prepare 1,000 g of coating solution. This coating solution has a solids content of about 30% (300 g solids/1,000 g total amount), and is a solution having an appropriate viscosity range [determined or estimated through experiments, for example: viscosity 500±100 cP]. The coating solution thus prepared has a solids ratio of GSE:FOS=1:1.

In the spraying-coating step (S250), the solution prepared in step S240 may be sprayed and coated onto a drum rotating at 15 rpm so that 30 wt % is attached relative to the total powder. Subsequently, in the vacuum drying step (S260), the powder coated in step S250 may be dried at 45 °C and −0.08 MPa for 1.5 hours to prepare a premix-coated powder having a moisture content of 4 wt % or less. Here, a vacuum dryer used for vacuum drying lowers the internal pressure to reduce the evaporation point of the solvent, thereby enabling rapid drying even at a relatively low temperature. In this embodiment, by treating at 45 °C and −0.08 MPa for 1.5 hours, destruction of the active components in the premix-coated powder is minimized, and the moisture content can be stabilized to 4 wt % or less.

In the low-temperature extraction step (S270), the coated powder of step S260 may be extracted for 60 minutes at 40 °C in a 50% ethanol-water mixed solvent under a liquid-to-solid ratio of 4 (v/w). In the reduced-pressure concentration step (S280), the extract of step S270 may be concentrated to 25 Brix solids under conditions of 45 °C and −0.08 MPa. In the spray-drying and re-mixing step (S290), the concentrate of step S280 is spray-dried under inlet 150 °C/outlet 75 °C conditions, and the resulting concentrated powder is re-mixed with the primary pulverized powder at a ratio of 1:9 (w/w) to prepare a low-temperature-extraction-re-mixed powder. Here, a spray dryer is equipment, or a drying method using such equipment, that produces powder by spraying the concentrate into high-temperature hot air to remove moisture in a short period of time.

Next, in the second fine-pulverizing step (S170), the re-mixed powder of step S160 may be finely pulverized to 100 mesh or less using a jet mill to uniformize the final particle size. Here, a jet mill is equipment that pulverizes particles by injecting high-pressure gas to cause the particles to collide and abrade each other, thereby generating little frictional heat and minimizing contamination. In this embodiment, the final particle size can be uniformized by micronizing to 100 mesh or less.

In the packaging step (S180), the second fine-pulverized powder may be filled into brown glass bottles in units of 120 g and sealed to prevent oxidation, moisture absorption, and photo-oxidation. In one embodiment, the packaging step (S180) may further include a step of blending additional materials such that the medicinal Platycodon root is about 92 wt %, glutinous rice is about 5 wt %, malt is about 1 wt %, grapefruit seed extract is about 1 wt %, and fructooligosaccharide is about 1 wt %.

The final formulation ratio of the Platycodon root active-component-enhanced powder prepared by applying the above-described process is shown in Table 1.

| | | Blending | Preferred |
|---|---|---|---|
| | | range | example |
| Raw material name | Specification | (w/w %) | (Examples 1-3) |
| Medicinal | 20 mesh or less | 85~95 | 92 |
| Platycodon root | | | |
| powder | | | |
| Glutinous rice | 80 mesh | 3~6 | 5 |
| powder, | | | |
| malt powder | 80 mesh | 0.5~3 | 1 |
| grapefruit seed | 10 Brix | 0~2 | 1 |
| extract | | | |
| fructooligosaccharide | Powder | 1~5 | 1 |

The samples were classified as follows according to whether each process step was performed.

Example 1: A case in which only the enzyme treatment step (S140) was performed, and the premix coating step (S150) and the low-temperature extraction and re-mixing step (S160) were omitted.

Example 2: A case in which the enzyme treatment step (S140) and the premix coating step (S150) were performed, but the low-temperature extraction and re-mixing step (S160) was omitted.

Example 3: A case in which the enzyme treatment step (S140), the premix coating step (S150), and the low-temperature extraction and re-mixing step (S160) were all performed consecutively.

Comparative Example: A case in which none of the three steps-enzyme treatment, coating treatment, and low-temperature extraction and re-mixing (S140, S150, S160)—were performed, and the method of preparing the comparative example was carried out by drying with hot air at 80 °C for 12 hours or more according to the conventional process, and then pulverizing to 40 mesh or less to obtain a powder.

According to this classification, quantitative analysis of active components and storage stability tests were performed on Examples 1-3 and the Comparative Example.

### Test Example 1: Quantification of Active Components (Unit: Mg/g, Inulin in %)

This test example is intended to confirm the effect of component enhancement at each process step by measuring the contents of the major active components (saponins, flavonoids, inulin, phytosterols) contained in Example 1, Example 2, Example 3, and the Comparative Example defined above. For each sample, 1 g was taken and reflux-extracted with 70% methanol, after which the solids were collected and analyzed. Total saponins were quantified by the HPLC-DAD method using platycodin D standard solution, flavonoids by the aluminum chloride colorimetric method (UV-Vis 415 nm), inulin by HPAEC-PAD, and phytosterols by GC-FID. All measurements were repeated three times and expressed as average values.

TABLE 2
Test Example 1-Quantification results of active components

| | | comparative | Example | Example | Example |
|---|---|---|---|---|---|
| Item | Unit | example | 1 | 2 | 3 |
| Total | mg/g | 6.8 ± 0.2 | 12.1 ± 0.4 | 11.9 ± 0.3 | 12.5 ± 0.4 |
| saponins | | | | | |
| Flavonoids | mg QE/g | 1.9 ± 0.1 | 5.7 ± 0.2 | 5.1 ± 0.2 | 5.4 ± 0.1 |
| Inulin | % | 1.2 ± 0.1 | 3.4 ± 0.1 | 2.9 ± 0.1 | 2.8 ± 0.1 |
| Phytosterols | mg/g | 0.7 ± 0.0 | 0.9 ± 0.0 | 1.0 ± 0.0 | 0.9 ± 0.0 |

Note) Values are mean±standard deviation (n=3). Example 3 shows an increase of more than 1.8-fold in total saponins, more than 2.8-fold in flavonoids, and more than 2.3-fold in inulin compared to the comparative example.

The results are summarized in Table 2. While the comparative example showed only 6.8 mg/g of total saponins, Example 1, to which only the enzyme treatment was applied, showed an increase to 12.1 mg/g, approximately 1.8-fold, and Example 2, in which premix coating was additionally applied, also maintained a similar level of 11.9 mg/g. In Example 3, in which all three steps (enzyme treatment-premix coating-low-temperature extraction and re-mixing) were performed consecutively, the saponin content was 12.5 mg/g, the highest. Flavonoids and inulin were also greatly influenced by the enzyme treatment step, with Example 1 showing the highest values of 5.7 mg QE/g and 3.4%, respectively, and Examples 2 and 3, in which subsequent processes were added, also maintaining values more than 2.5 times higher than those of the comparative example. Phytosterols increased slightly to 1.0 mg/g in Examples 2 and 3, which contained grapefruit seed extract.

When the test results are combined as described above, Example 3, to which the three-step continuous process was applied, exhibited the most superior total saponin content, and it can be confirmed that the enzyme treatment step played a key role in the increases in flavonoids and inulin attributable to the cell wall decomposition effect. Therefore, it can be seen that the three-step continuous process of the present invention is the most effective method for simultaneously maximizing the functional components of the medicinal Platycodon root powder.

### Test Example 2-Storage Stability (40 °C.75% RH, 6 Months)

Test Example 2 is intended to confirm how well the manufactured powder product maintains appearance quality and microbial safety when stored for a long period under high-temperature and high-humidity conditions. For this purpose, 100 g each of the samples of the comparative example and Examples 1, 2, and 3 were placed in heat-resistant glass bottles with an inner diameter of 5 cm, sealed, and stored for 6 months in an incubator under conditions of 40 °C and 75% relative humidity. At the end of the storage period, caking, color difference (ΔE), and total bacterial count were sequentially measured. Caking was evaluated by the finger-compression method and determined in four grades: “none (−), slight (+), moderate (++), severe (+++).” The color difference was calculated as ΔEab by measuring the Lab* values before and after storage with a portable spectrocolorimeter. For the total bacterial count, 1 g of sample was suspended in 9 ml of sterile physiological saline, spread onto a standard plate medium, incubated at 37 °C for 48 hours, and the number of colonies was counted.

TABLE 3

| | | | Total |
|---|---|---|---|
| | | Color | bacterial |
| | | difference | count |
| Sample | Caking | ΔE | (cfu/g) |
| Comparative | Severe | 12.4 | 4.6 × 10⁴ |
| Example | (+++) | | |
| Example 1 | None(−) | 2.9 | 9.0 × 10² |
| Example 2 | None(−) | 3.0 | 6.5 × 10² |
| Example 3 | None(−) | 3.1 | 8.2 × 10² |

The results are shown in Table 3. In the comparative example, which used only the conventional hot-air drying process, a large amount of caking formed on the entire surface of the sample after 6 months of storage (“+++”), and the color difference also increased significantly to ΔE 12.4, showing clearly visible browning. In contrast, Example 1, in which only the enzyme treatment step was applied, showed no caking at all, and the color difference was minimal at ΔE 2.9. Example 2, in which premix coating was added, and Example 3, in which all three steps were performed, also showed no caking, and the ΔE values were 3.0 and 3.1, respectively, maintaining levels similar to Example 1. In terms of microorganisms, the comparative example exceeded the limit for dried foods under the Food Code (10⁴ cfu/g), showing 4.6×10⁴ cfu/g, whereas Examples 1, 2, and 3 were all 103 cfu/g or less, with Example 2 containing grapefruit seed extract showing the lowest value at 6.5×10² cfu/g.

As described above, even the enzyme treatment alone greatly suppressed caking and discoloration, and when premix coating was additionally applied, antimicrobial effects were added, resulting in the highest storage stability. Example 3, which further included the low-temperature extraction and re-mixing step, also enhanced intrinsic components while maintaining appearance and microbial stability comparable to Example 2. Therefore, the stepwise process of the present invention has been proven to be effective for long-term quality preservation of medicinal Platycodon root powder products.

Meanwhile, although the detailed description of the present disclosure has described specific embodiments, it is of course that various modifications are possible without departing from the scope of the present disclosure. Therefore, the scope of the present disclosure should not be limited and defined by the described embodiments, but should be defined by the claims described below and by equivalents to the scope of the claims.

The description of the presented embodiments is provided so that a person of ordinary skill in the technical field of the present invention may utilize or practice the present invention. Various modifications to these embodiments will be apparent to a person of ordinary skill in the technical field of the present invention, and the general principles defined herein may be applied to other embodiments without departing from the scope of the present invention. Accordingly, the present invention is not limited to the embodiments presented herein, but should be interpreted in the broadest scope consistent with the principles and novel features presented herein.

What is claimed is:

1. A method for preparing a powder comprising Platycodon grandiflorus, the method comprising:
(a) washing and dehydrating a Platycodon raw material and then hot-air drying the raw material at 45 °C or lower to adjust the moisture content to 10 wt % or less;
(b) performing a first pulverization of the dried Platycodon raw material to 20 mesh or less; and
(c) mixing the primary powder and distilled water at 1:3 (w/v) to prepare a slurry, adding 0.5 wt % cellulase and 0.05-0.5 wt % pectinase, reacting under conditions of 45-55 °C and pH 4.5-5.5 for 60-120 minutes, and freeze-drying under conditions of −40 °C and 10 Pa to obtain an enzyme-treated powder having a moisture content of 5 wt % or less,
wherein the method comprises an enzyme treatment step.

2. The method of claim 1, further comprising:
a premix coating step in which, after the enzyme treatment step, a premix solution prepared by mixing grapefruit seed extract and fructooligosaccharide at a solids ratio of 1:1 (w/w) and homogenizing the mixture at 45 °C is sprayed and coated onto a drum rotating at 15 rpm, and then vacuum-dried at 45 °C and −0.05 to −0.1 MPa for 1 to 2 hours to produce a premix-coated powder.

3. The method of claim 2, further comprising:
a low-temperature extraction and re-mixing step in which, after the premix coating step, the premix-coated powder is extracted with a 50% ethanol-water mixed solvent at 35-45 °C for 30-60 minutes, the extract is concentrated under reduced pressure at 40-50 °C and −0.05 to −0.09 MPa until the solids content reaches 20-30 Brix, and the concentrated powder obtained by drying with a spray dryer (inlet 140-160 °C/outlet 70-80 °C) is re-mixed with the original powder at a ratio of 1:9 (w/w).

4. The method of claim 3, comprising:
(d) after the low-temperature extraction and re-mixing step, performing a second fine-pulverizing step in which the re-mixed powder is secondarily pulverized with a jet mill to 80 mesh or more and 150 mesh or less to uniformize the particle size; and
(e) a packaging step in which the second powder is blended such that the medicinal Platycodon root is 85-95 wt %, glutinous rice is 3-6 wt %, malt is 0.5-3 wt %, grapefruit seed extract is 0.5-2 wt %, and fructooligosaccharide is 1-5 wt %, and then filled and sealed in brown glass bottles, wherein the method is a method for preparing a medicinal Platycodon root powder.

5. A medicinal Platycodon root syrup comprising 1 wt % or more and 20 wt % or less of the medicinal Platycodon root powder prepared by the method of claim 3 or claim 4, comprising isomaltooligosaccharide syrup and grain syrup as the remainder, and having a moisture content adjusted to 25 wt % to 35 wt % through a heating and concentration process,
a medicinal Platycodon root syrup.
